(19) United States
(12) Patent Application Publication
(10) Pub. No.: US 2004/0087957 A1
(43) Pub. Date: May 6, 2004

(54) Bone removal device and method of use

(75) Inventor: Alex Kunzler, Issaquah, WA (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE

(21) Appl. No.: 10/696,450

(22) Filed: Oct. 29, 2003

Related U.S. Application Data
(63) Continuation-in-part of application No. 10/294,502, filed on Nov. 14, 2002.
(60) Provisional application No. 60/332,111, filed on Nov. 16, 2001.

(51) Int. Cl. A61B 17/00

(52) U.S. Cl. 606/79

(57) ABSTRACT

A bone preparation device, comprises a guide body, a bone removal device having a longitudinal axis extending between a proximal portion and a distal portion, a pair of guide members movably engaged between said guide body and said bone removal device, and an alignment device movably engaged between said guide members. The bone removal device may be movably guided by the guide members and the alignment device with respect to said guide body through a predetermined pattern.

### CROSS REFERENCE

This application is a continuation-in-part of U.S. patent application Ser. No. 10/294,502, filed on Nov. 14, 2002, having the title “Bone Removal Device and Method of Use,” which claims the benefit of U.S. Provisional Application No. 60/332,111, filed Nov. 16, 2001.

### FIELD OF THE INVENTION

The present invention relates generally to medical procedures and apparatus for use during surgery, and more particularly, to an apparatus and method for positioning and controlling the movement of a bone removal device.

### BACKGROUND

Joint replacement surgery often may require the preparation of the end of a bone to receive a joint prosthesis. In particular, a device may be used to remove bone in the vicinity of the joint. Current instruments used for bone removal may be difficult to position and their movement may be difficult to guide. As a result, the profile of the material removed by current devices may be difficult to control.

### SUMMARY

In one embodiment, a bone preparation device comprises a guide body or cage, a bone removal device having a longitudinal axis extending between a proximal portion and a distal portion, a pair of guide members movably engaged between said guide body and said bone removal device, and an alignment device movably engaged between said guide members. The bone removal device may be movably guided by the guide members and the alignment device with respect to said guide body through a predetermined pattern.

In another embodiment, the alignment device comprises a gear.

In another embodiment, the alignment device comprises a connecting rod.

In another embodiment, the alignment device comprises a band connected between a pair of pulleys.

In still another embodiment, a method of bone preparation comprises moving a bone removal device at least partially housed within a guide body. A pair of guide members are movably engaged between the guide body and the bone removal device and rotate with the movement of the bone removal device. The method further comprises guiding the movement of the bone removal device through a predetermined pattern and aligning the rotation of the guide members with an alignment device movably engaged between the guide members.

### BRIEF DESCRIPTION OF THE DRAWINGS

FIG. 1A is a partial cross sectional schematic plan view of a bone removal device in accordance with a first embodiment of the present invention.

FIG. 1B is a partial cross sectional schematic plan view of a bone removal device having an alignment device in accordance with a second embodiment of the present invention.

FIG. 1C is a partial cross sectional schematic plan view of a bone removal device having an alignment device in accordance with a third embodiment of the present invention.

FIG. 1D is a partial cross sectional schematic plan view of a bone removal device having an alignment device in accordance with a fourth embodiment of the present invention.

FIG. 2 is a top view of the bone removal device of FIG. 1A taken along the line 2-2.

FIG. 3 is a perspective view of a guide mechanism utilized in the bone removal device illustrated in FIG. 1A.

FIG. 4 is a partial cross sectional schematic plan view of an alternative embodiment of a bone removal device in accordance with the present invention.

FIG. 5 is a top view of the bone removal device of FIG. 4 taken along the line 5-5.

FIG. 6 is a partial cross sectional schematic plan view of yet another alternative embodiment of a bone removal device in accordance with the present invention.

FIG. 7 is a top view of the bone removal device of FIG. 6 taken along the line 7-7.

FIG. 8A is a side view of the distal end of the bone removal device shown in FIG. 1A.

FIG. 8B is a side view of a prosthesis that may be implanted into an opening prepared using the bone removal device of FIG. 8A.

FIG. 9A is a schematic view of an alternative embodiment of the bone removal device of the present invention.

FIG. 9B is a profile of a bone removal element for use with the bone removal device of FIG. 9A.

FIG. 9C shows a bone removal profile created by the bone removal element of FIG. 9B utilized with the bone removal device of FIG. 9A.

FIG. 10A is a schematic view of an alternative embodiment of the bone removal device of the present invention.

FIG. 10B is a profile of a bone removal element for use with the bone removal device of FIG. 10A.

FIG. 10C shows a bone removal profile created by the bone removal element of FIG. 10B utilized with the bone removal device of FIG. 10A.

FIG. 11A is a schematic view of an alternative embodiment of the bone removal device of the present invention.

FIG. 11B is a profile of a bone removal element for use with the bone removal device of FIG. 11A.

FIG. 11C shows a bone removal profile created by the bone removal element of FIG. 11B utilized with the bone removal device of FIG. 11A.

FIG. 12A is a schematic view of an alternative embodiment of the bone removal device of the present invention.

FIG. 12B is a profile of a bone removal element for use with the bone removal device of FIG. 12A.

FIG. 12C shows a bone removal profile created by the bone removal element of FIG. 12B utilized with the bone removal device of FIG. 12A.

FIG. 13A is a schematic view of an alternative embodiment of the bone removal device of the present invention.

FIG. 13B is a profile of a bone removal element for use with the bone removal device of FIG. 13A.

FIG. 13C shows a bone removal profile created by the bone removal element of FIG. 13B utilized with the bone removal device of FIG. 13A.

FIG. 14A is a schematic drawing of a bone removal profile created by an embodiment of a bone removal device of the present invention.

FIG. 14B is a schematic drawing of the complementary profile of a portion of the prosthesis implanted within that bone removal profile in accordance with a method of the present invention.

FIG. 15 is a cross sectional view of a prosthesis that may be implanted into an opening prepared using the bone removal device of the present invention.

FIG. 16 is a cross sectional schematic view of a bone removal device in accordance with the present invention, and in particular, illustrates a unique drive mechanism.

### DETAILED DESCRIPTION

The present invention relates to a unique bone removal device which may be used for preparing the end of a bone to receive a joint prosthesis. For example, the device may be used to prepare one or more vertebral bodies to receive an intervertebral prosthesis. In particular, the device may include a bone removal tool movably mounted to a support structure or cage. The bone removal tool may include a housing having a proximal end and a distal end. A drive mechanism is contained within the housing. The internal design of the bone removal tool of the present invention may be the same as the designs described in U.S. Pat. No. 6,562,045 filed on Aug. 22, 2001, entitled “Machining Apparatus,” the entire contents of which are incorporated herein by reference.

A bone removal element may be mounted at the distal end of the housing. The proximal end of the housing may be adapted to be attached to a drive source to drive the drive mechanism. A pair of guide mechanisms that interconnect the bone removal tool and the support structure may be positioned between the proximal end and the distal end of the housing. The guide mechanisms may be designed to allow the user to guide the movement of the device, and in particular to guide the movement of the bone removal element in order to create a specific shape or profile within or in the end of the bone.

In accordance with one embodiment, the guide mechanisms may consist of one or more offset shafts. Each shaft has a first guide pin and a second guide pin associated therewith. The first guide pin may be rotatably attached to the support structure. The second guide pin may be rotatably mounted on the bone removal tool. An alignment device may be movably engaged between the pair of guide mechanisms to maintain the guide mechanisms in alignment such that each guide member moves at approximately the same speed and in approximately the same direction. In use, the bone removal tool may be manipulated by the user such that the first guide pin and the second guide pin rotate about one another, and thereby guide the motion of the bone removal element along a predetermined path.

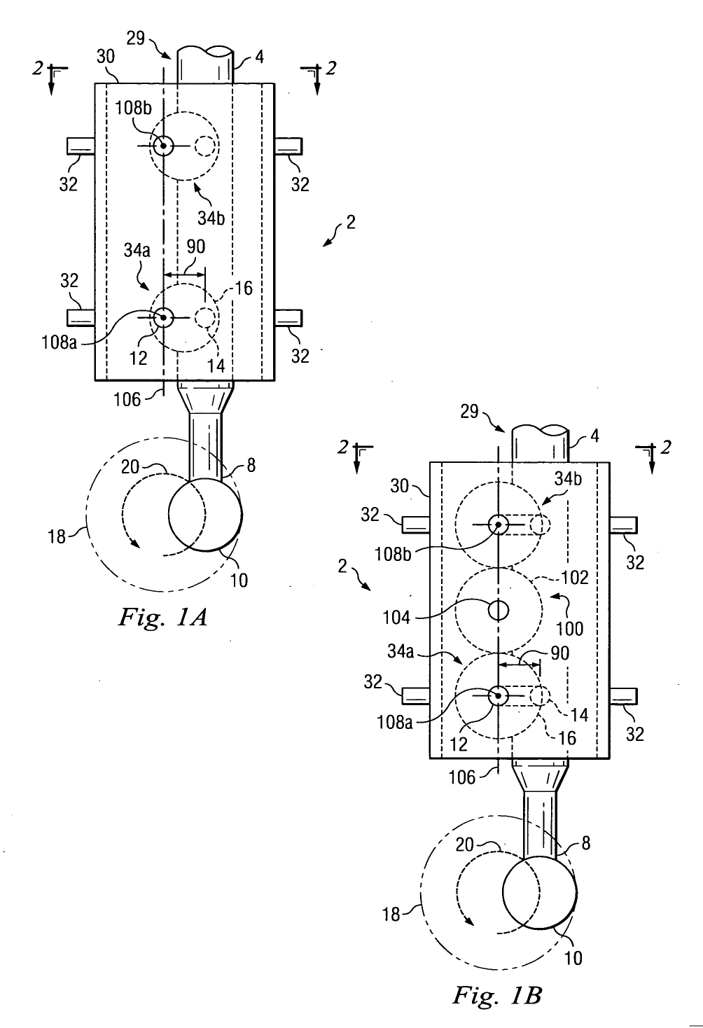

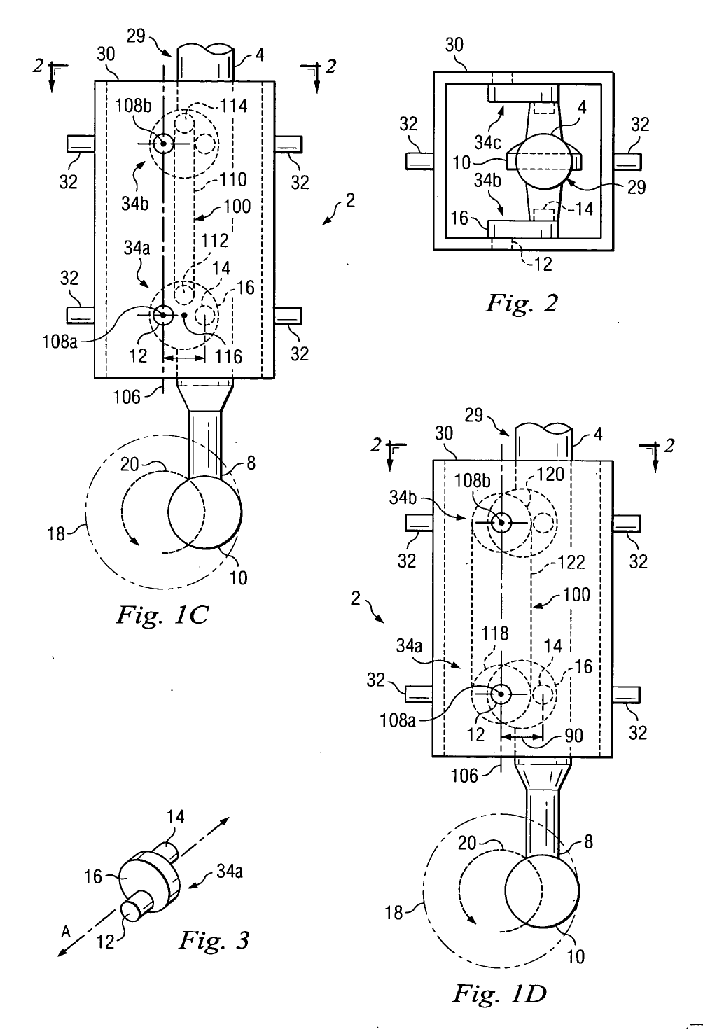

Referring now to the drawings, various embodiments of the present invention are illustrated. Referring to FIGS. 1A and 2, bone removal device 2 is shown. The device 2 includes a bone removal tool 29 and a cage 30. Bone removal tool 29 includes housing 4 having a proximal end (not shown) and a distal end 8. A bone removal element 10 is positioned at the distal end 8 of the housing 4. Bone removal element 10 may incorporate a variety of mechanisms to remove bone. For example, bone removal element 10 may include a mechanical mechanism to remove bone such as a cutting edge, an abrasive surface, or a combination thereof. Alternatively, bone removal element may include a tissue obliteration mechanism such as an electron or RF beam, ultrasound, or fluid jet cutting.

Cage 30 includes at least one set of pivot pins 32. The pivot pins 32 are adapted to interface with a machining jig or scaffold (not shown) similar to the scaffold designs described in co-pending U.S. patent application Ser. No. 09/923,891 filed on Aug. 7, 2001 entitled “Method and Apparatus for Stereotactic Implantation,” the entire contents of which is incorporated herein by reference.

Cage 30 is movably attached to the bone removal tool 29 via a guide mechanism 34a. Additional guide mechanisms such as 34b (FIG. 1A), 34c (FIG. 2), and 34d (not shown, but located on the opposite side of the bone removal tool 29 from the guide mechanism 34a and beneath 34c) may also be employed. Because these additional guide mechanisms may be similar or identical to guide mechanism 34a, they will not be described in detail.

In accordance with one embodiment, guide mechanism 34a may be configured as an offset shaft. As seen best in FIG. 3, guide mechanism 34a includes first and second guide pins 12 and 14 interconnected by body 16. The axis of the first and second guide pins 12 and 14 are substantially parallel, but may not be congruent to one another. As best illustrated in FIG. 2, first guide pin 12 is rotatably attached to cage 30. Second guide pin 14 is rotatably attached to housing 4. Alternatively, guide pins 12, 14 could be rotatably attached to body 16, and non-movably attached to cage 30 and/or housing 4. A center line 106 (FIG. 1A) may extend between a pair of pivot points 108a and 108b of the guide mechanisms 34a and 34b, respectively.

In use, housing 4 is moved such that guide pin 14 causes body 16 to rotate about an axis A (see FIG. 3). As body 16 rotates, bone removal element rotates along arrow 20 to create a first bone removal profile 18 (see FIG. 1A) within a first plane.

Referring now to FIG. 1B, in order to keep guide mechanism 34a and 34b in alignment while rotating, an alignment device 100 may extend between guide mechanism 34a and 34b. In accordance with this embodiment, the alignment device 100 may be located within the cage 30. In other embodiments, the device 100 may be located outside the cage 30 or within an opening in cage 30. In one embodiment, as shown in FIG. 1B, the alignment device 100 comprises a toothed gear 102, such as an idler or connecting gear. The gear 102 may engage guide mechanisms 34a and 34b and may be rotatably attached to cage 30 with a guide pin 104. To permit engagement with the gear 102, the guide mechanisms 34a and 34b may be toothed. In this embodiment, as the guide mechanism 34a rotates, gear 102 may rotate at the same speed and in the opposite direction as guide mechanism 34a which can cause guide mechanism 34b to rotate at the same speed and in the same direction as guide mechanism 34a. Thus, the center line 106 extending between the pivot point 108a and the pivot point 108b can be maintained in relatively parallel alignment with the housing 4. An alignment device similar or identical to alignment device 100 of this embodiment may also be configured to extend between guide mechanisms 34c and 34b.

In another embodiment, as shown in FIG. 1C, the alignment device 100 comprises a connecting rod 110 which may extend between guide mechanisms 34a and 34b. The connecting rod 110 may be rotatably attached to guide mechanisms 34a and 34b with guide pins 112 and 114, respectively. The guide pin 112 may attach to the guide mechanism 34a at a location such that the guide pins 112, 12, and 14 may be equidistant from the radial center 116 of guide mechanism 34a with guide pin 112 equidistant from points 12 and 14. The guide pin 114 can be attached at an equivalent location to guide mechanism 34b. In accordance with this embodiment, as the guide mechanism 34a rotates, the connecting rod 110 can permit the guide mechanism 34b to rotate at the same speed and in the same direction. Thus, the center line 106 extending between the pivot points 108a and 108b can be maintained in relatively parallel alignment with the housing 4. An alignment device similar or identical to alignment device 100 of this embodiment, may be engaged with guide mechanisms 34c and 34d.

In still another embodiment, as shown in FIG. 1D, the alignment device 100 may comprise a pulley 118 which may be concentric with and fixedly attached to pin 12 of guide mechanism 34a. The pulley 118 may rotate with pin 12 around pivot point 108a. The alignment device 100 may also comprise a pulley 120 attached in substantially the same manner to guide mechanism 34b. The alignment device 100 may further comprise a band 122 which may extend around pulleys 118 and 120. The pulleys 118 and 120 may be, for instance, sprockets or timing pulleys. The band 122 may be, for example, a chain or a timing belt. The band 122 may extend over both pulleys 118 and 120. In this embodiment, as the guide mechanism 34a rotates, the band 122, engaged with the pulleys 118 and 120, can cause the guide mechanism 34b to rotate at the same speed and in the same direction. Thus, a center line 106 extending between pivot points 108a and 108b of the guide mechanisms 34a and 34b, respectively, can be maintained in relatively parallel alignment with the housing 4. An alignment device similar or identical to alignment device 100 of this embodiment, may be engaged with guide mechanisms 34c and 34d.

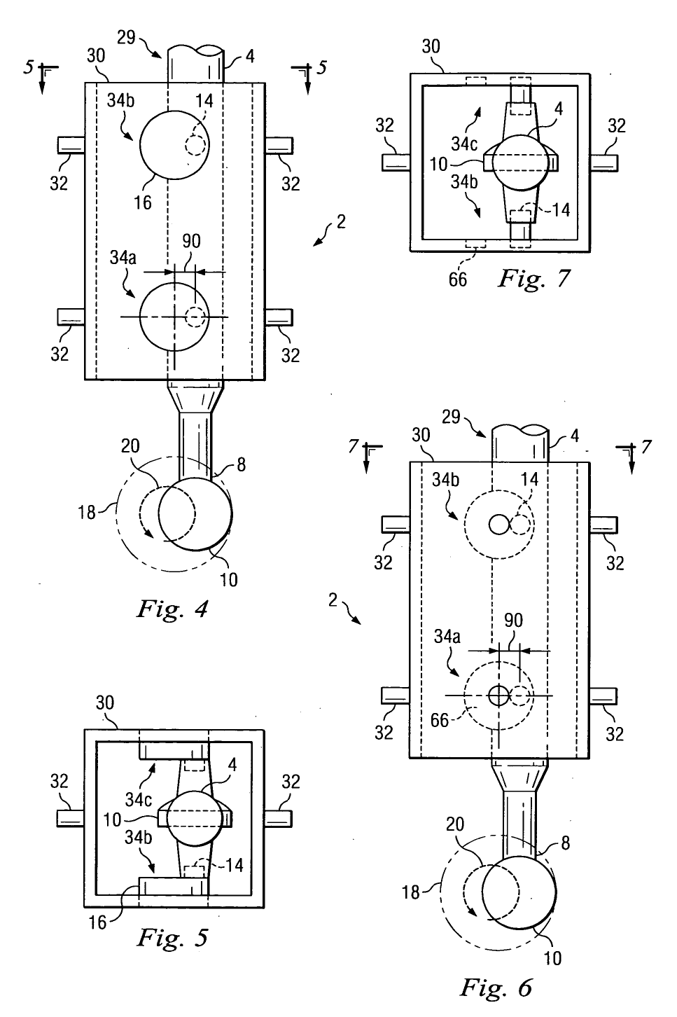

FIGS. 4 and 5 illustrate an alternative embodiment of guide mechanism 34a. In accordance with this embodiment, guide mechanism 34a consists of a body 16 rotatably positioned within an opening in cage 30 and a pin 14 rotatably attached to housing 4. As with the previous embodiment, as body 16 rotates, bone removal element rotates along arrow 20 to create a first bone removal profile 18 within a first plane.

FIGS. 6 and 7 illustrate yet another embodiment of guide mechanism 34a. In accordance with this embodiment, the guide mechanism 34a consists of a track 66 within the side of cage 30. Guide pin 14 is positioned within track 66. Housing 4 may be moved such that pins 14 rotate about tracks 66 thereby causing bone removal element to rotate along arrow 20 to create first bone removal profile 18 within a first plane.

Any of the alignment devices 100 described in the above embodiments 1B-1D may be adapted to operate with the embodiments described in FIGS. 4-7. To function within the embodiments of FIGS. 4-7, the alignment device 100 may be located within the cage 30, within an opening in the cage 30, or outside the cage 30.

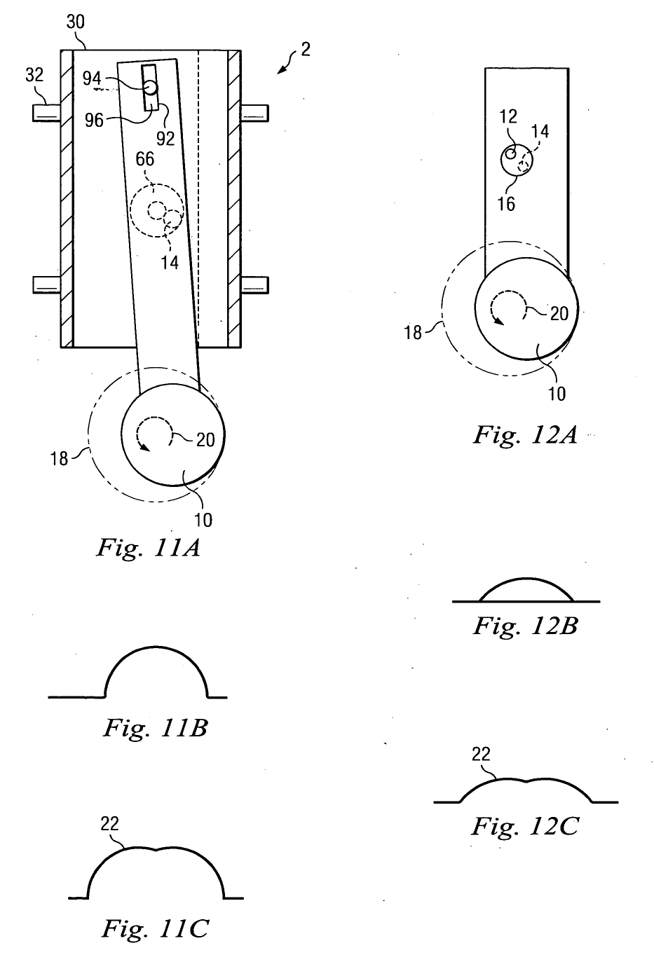

In the embodiments show in FIGS. 1-7, guide mechanisms 34a and 34b are shaped to create a circular bone removal profile 18. Those skilled in the art will appreciate that the shape of guide mechanism 34a and 34b can be modified to create a bone removal profile 18 having any one of a variety of shapes. For circular bone removal profiles 18, the radius of the profile is determined by the radius of the bone removal element 10 and the radius of the path 20 of the center of the bone removal element. The radius of path 20 is determined by the critical radius dimension 90 of the guide mechanism 34a as illustrated in FIG. 1A, FIG. 4 and FIG. 6. In addition, each of the embodiments illustrated in the drawings includes two guide mechanisms on two sides of device 2. Alternatively, the device could include only one guide mechanism on one side of device 2, one guide mechanism on two sides of device 2, two guide mechanisms on one side of device 2, or a plurality of guide mechanisms on one or more sides of device 2. As illustrated in FIGS. 9-11, in those embodiments where only one guide mechanism 34a is included on one side of a device 2, a stabilizing structure 92 may be provided to facilitate guiding bone removal element 10 along profile 18. Stabilizing structure 92 may include a pin 94 attached to housing 4 positioned within a slot 96 within support structure 30.

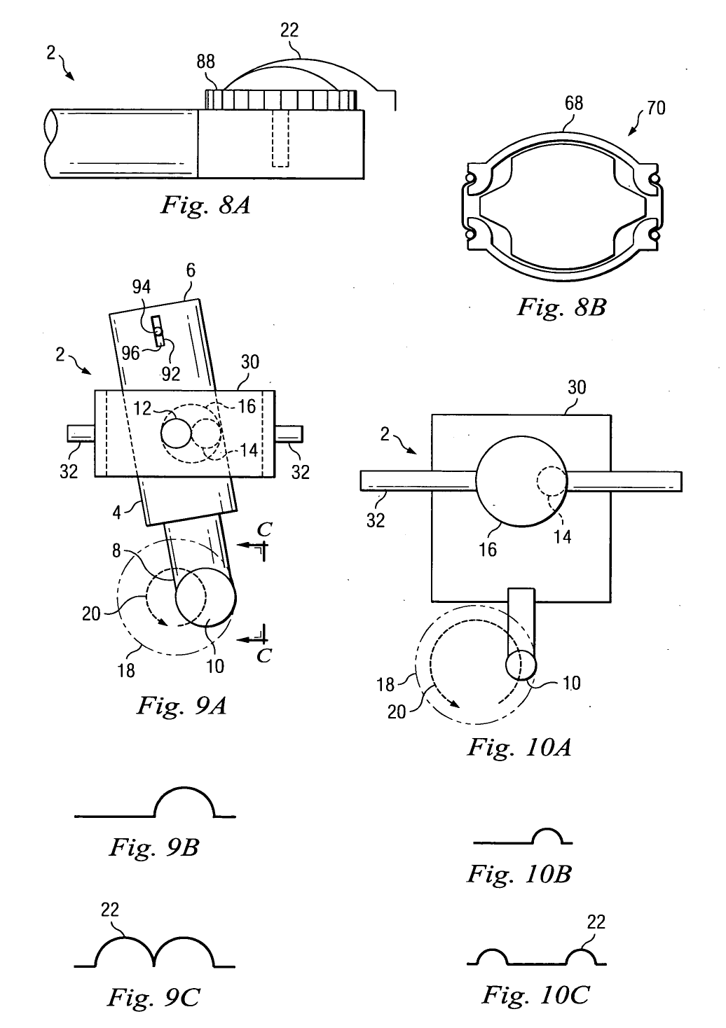

FIG. 8A is a lateral schematic view of the proximal end of device 2 shown in FIG. 1A, and illustrates a second bone removal profile 22 within a second plane that is substantially perpendicular to the first plane in which bone removal profile 18 lies. The bone removal device creates the second bone removal profile 22 as body 16 of guide mechanism 34a rotates about its axis. FIG. 8B illustrates the exterior profile 68 of a joint prosthesis 70. In accordance with the present invention, the exterior profile 68 of joint prosthesis 70 substantially matches the second bone removal profile 22. Similarly, although not illustrated in the drawings, the profile of the prosthesis along the first plane substantially matches the first bone removal profile 18.

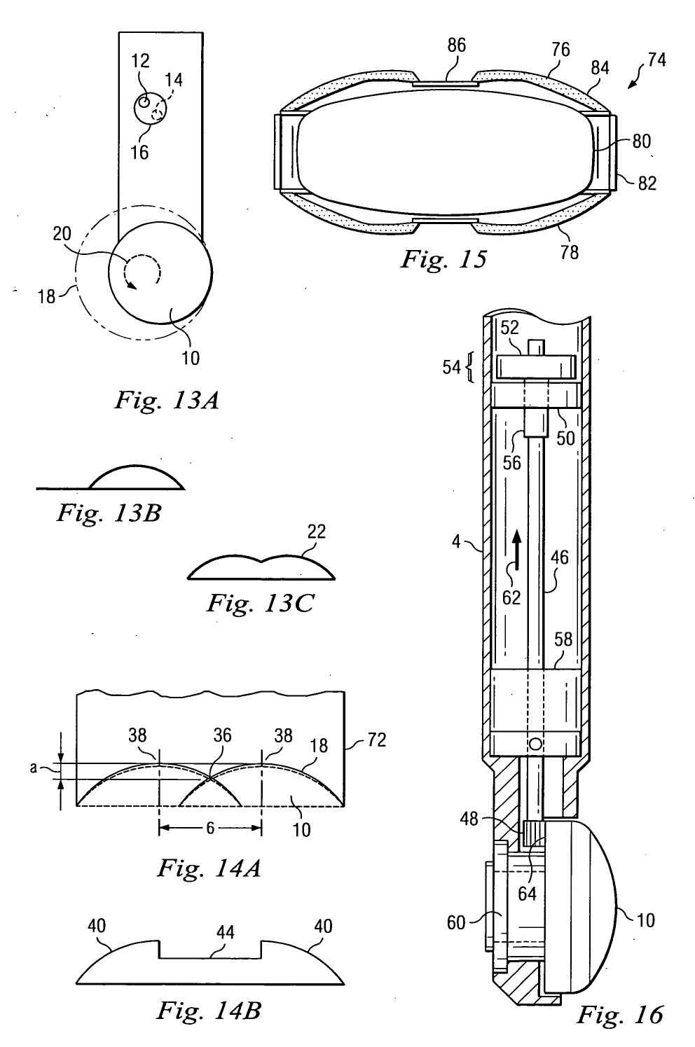

FIGS. 9A-13C illustrate alternative embodiments of the present invention. In each of these Figures. FIGS. 9A, 10A, 11A, 12A, and 13A represent a schematic drawing of a bone removal device 2 in accordance with the present invention. FIGS. 9B, 10B, 11B, 12B, and 13B represents the lateral profile of the bone removal element 10 shown in the corresponding figure A. FIGS. 9C, 10C, 11C, 12C, and 13C represent the second bone removal profile 22 created by the device shown in the corresponding figure A when used with the bone removal device 2 of the corresponding figure B. For ease of illustration the support structure or cage is not shown in all embodiments. One skilled in the art will appreciate from these figures that the device of the present invention may create a variety of profiles within a bone surface. The exact profile will be determined by: 1) the size and shape of the guiding mechanism; and the size and shape of the bone removal element within both the first and the second planes. As noted above, although the guide mechanism is illustrated as being essentially circular, it (as well as the shape of the bone removal element along the first and second planes) may also be elliptical, square, hexagonal, or any other polygonal shape, or may include shapes that are only partially arcuate or formed from a plurality of arcs with different radii of curvature. Preferably, the bone removal element has a profile along the second plane that consists of two merged profiles that are mirror images of one another. An example of such a profile is shown in FIG. 13B. In this embodiment the bone removal element has a non-circular profile that consists of two merged mirrored arcs, whereby each arc has the same radius of curvature. The bone removal element profile may also include a shoulder 88 as shown in the device illustrated in FIG. 8A. One skilled in the art will further appreciate that a prosthesis may be provided having an outer surface that substantially matches or complements the profile created by the bone removal device 2.

FIGS. 14A and 14B illustrate an outer surface of a possible prostheses design and its complementary bone removal profile. As shown in FIG. 14A, bone removal element 10 (whose positions are illustrated by the dotted lines) rotates as described above to create a bone removal profile 18 within a bone 72. In accordance with this embodiment, a central protruding bone portion 36 is created by the bone removal element 10. The protruding portion 36 extends between the two apexes 38 of the bone removal profile 18, and has a width of 6 and a height of a. As shown in FIG. 14B, the complimentary prosthesis surface profile includes an outer radial section 40 and a central recessed section 44 designed to accommodate the bone protrusion 36. In particular, recessed section 44 has a width that is greater than b and a height that is greater than a. Outer radial section 40 has substantially the same radius of curvature as a portion of the bone remove element 10 and the bone removal profile 18 created thereby.

FIG. 15 is a cross sectional view of a spinal disc prosthesis 74 that may be implanted in accordance with the present invention. Prosthesis 74 is similar in design to the devices described in co-pending U.S. patent application Ser. No. 09/924,298 filed on Aug. 8, 2001 entitled “Implantable Joint Prosthesis,” and which is incorporated herein by reference. Prosthesis 74 includes an upper shell 76, a lower shell 78 and a central component 80 positioned between the two shells. The prosthesis 74 further includes an annular sheath 82 that is attached to the upper and lower shells 76, 78 and surrounds the central component 80. Sheath 82 seals the central component from the external environment. A portion of the outer surface of the shells 76, 78 includes a bone ingrowth surface 84. Preferably, the bone ingrowth surface 84 is a porous coating. As shown in FIG. 15, each of the shells 76, 78 of prosthesis 74 includes a central recessed portion 86 defined by the absence of the porous coating. In accordance with the present invention, a bone removal device having a bone removal element profile similar to that shown in FIG. 14A is provided to prepare a vertebral endplate to receive prosthesis 74. In accordance with this embodiment of the present invention, the bone removal element profile is sized and shaped such that a protrusion 36 having a height and width that is smaller than the height and width of the recessed portion 86 of prosthesis 74.

FIG. 16 is a schematic cross sectional view of a bone removal device in accordance with the present invention, and illustrates a unique drive mechanism. The drive mechanism includes a drive shaft 46 having gears 48 located at its distal end that are adapted to interface with gears 64 located on the under surface of bone removal element 10. The proximal end (not shown) of drive shaft 46 is adapted to be attached to a standard power source such as, for example, by means of any well-known mechanical interlocking coupling. The housing 4 of the device includes a proximal support member 50 through which drive shaft 46 extends. Drive shaft 46 includes a rotation-facilitating segment 52 having an expanded section 54 and a support member interface section 56. In accordance with a preferred embodiment of the present invention, drive shaft 46 is made from a biocompatible material such as, for example, a biocompatible metal or a biocompatible polymer. Preferably, drive shaft 46 is made from stainless steel. Rotation facilitating segment 52 is preferably made from a wear resistant ceramic or polymer, such as a Teflon impregnated Delrin. In addition, the support members 50 are desirably made from stainless steel. To further facilitate rotation of shaft 46 a bushing 58, which may be made of ceramic or other suitable bushing material, is mounted along the shaft within housing 4. In addition, bone removal element 10 is mounted on a similar bushing 60 to facilitate its movement. In use, this design provides a disposable drive shaft 46 that can be quickly and easily removed and replaced when the gears 48 become worn. Shaft 46 is removed from housing 4 by pulling its proximal end in the direction of arrow 62. When a new shaft 46 is inserted, expanded section 54 provides a stop mechanism against support members 50 to properly position shaft 46 along the longitudinal axis of the device. When a motor or drive mechanism is coupled to the proximal end of the device it locks shaft 46 in place. Although the gearing arrangement is illustrated as a spur gear-type pinion on shaft 46 and a face gear on one surface of the bone removal element 10, other gearing arrangements are possible, so long as the gear on shaft 46 is removable through the apertures in housing 4, and is easily inserted and coupled with the other gears in the arrangement. Possible examples include bevel gears, or a face gear on the end of shaft 46 that couples with gearing around the edge of bone removal element 10.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

What is claimed is:

1. A bone preparation device, comprising:
a guide body;
a bone removal device having a longitudinal axis extending between a proximal portion and a distal portion;
a pair of guide members movably engaged between said guide body and said bone removal device; and
an alignment device movably engaged between said guide members,
wherein said bone removal device may be movably guided by said guide members and said alignment device with respect to said guide body through a predetermined pattern.

2. The bone preparation device of claim 1, wherein said guide members are rotatably connected to said bone removal device.

3. The bone preparation device of claim 2, wherein said guide members are pivotally connected to said bone removal device.

4. The bone preparation device of claim 1, wherein said predetermined pattern is circular.

5. The bone preparation device of claim 4, wherein said bone removal device forms a generally toroidal shape in the bone.

6. The bone preparation device of claim 1, wherein each of said guide members has a major side surface substantially parallel to said longitudinal axis and each of said guide members is joined to said major side surface.

7. The bone preparation device of claim 6, wherein said predetermined pattern is defined in a plane substantially parallel to said longitudinal axis.

8. The bone preparation device of claim 1, wherein each of said guide members is rotationally connected to said guide body and rotationally connected to said bone removal device.

9. The bone preparation device of claim 1, wherein said guide body is a cage at least partially encircling said bone removal device.

10. The bone preparation device of claim 9, wherein said body has an inner surface, said inner surface having at least one track, said guide members following said at least one track to control said bone removal device to form said predetermined pattern.

11. The bone preparation device of claim 10, wherein said guide members are pivotally connected to said bone removal device.

12. The bone preparation device of claim 1, wherein each of said guide members includes a first projection pivotally engaged to said bone removal device.

13. The bone preparation device of claim 12, wherein each of said guide members includes a second projection in substantial opposition to said first projection, said second projection pivotally engaged with said guide body.

14. The bone preparation device of claim 1, wherein said bone removal device includes a bone removal element disposed adjacent said distal end, said bone removal element having an axis of rotation, said axis of rotation offset with respect to said longitudinal axis.

15. The bone preparation device of claim 14, wherein said axis of rotation is substantially transverse to said longitudinal axis.

16. The bone preparation device of claim 1, wherein said bone removal device is coupled to a power source adjacent said proximal end.

17. The bone preparation device of claim 1, wherein said guide members are configured to simultaneously control axial displacement of said bone removal device with respect to said guide body and movement transverse to said longitudinal axis to generate a substantially non-linear predetermined pattern.

18. The bone preparation device of claim 1, wherein said alignment device controls the speed and direction of said guide members.

19. The bone preparation device of claim 1, wherein said alignment device comprises a gear.

20. The bone preparation device of claim 1, wherein said alignment device comprises a connecting rod.

21. The bone preparation device of claim 1, wherein said alignment device comprises a band connected between a pair of pulleys, wherein each pulley is connected to a respective guide member.

22. The bone preparation device of claim 1, wherein said alignment device is adapted to move each guide member at approximately the same speed.

23. The bone preparation device of claim 1, wherein said alignment device is adapted to move each guide member in approximately the same direction

24. The bone preparation device of claim 1, wherein said alignment device is positioned within said guide body.

25. A method of bone preparation, the method comprising:
moving a bone removal device at least partially housed within a guide body, wherein a pair of guide members are movably engaged between the guide body and the bone removal device and rotate with the movement of the bone removal device;
guiding the movement of the bone removal device through a predetermined pattern;
aligning the rotation of the guide members with an alignment device movably engaged between the guide members.

26. The bone preparation method of claim 25 wherein the alignment device comprises a gear.

27. The bone preparation method of claim 25 wherein the alignment device comprises a connecting rod.

28. The bone preparation method of claim 25 wherein the alignment device comprises a band connected between a pair of pulleys, wherein each pulley is connected to a respective guide member.

29. The bone preparation method of claim 25 wherein the alignment device is adapted to move each guide member at approximately the same speed.

30. The bone preparation method of claim 25 wherein the alignment device is adapted to move each guide member in approximately the same direction.

31. A vertebral body preparation device, comprising:
a guide body;
a bone removal device having a longitudinal axis extending between a proximal portion and a distal portion;
a pair of guide members movably engaged between said guide body and said bone removal device; and
an alignment device movably engaged between said guide members, wherein said bone removal device may be movably guided by said guide members and said alignment device with respect to said guide body through a predetermined pattern.

32. The vertebral body preparation device of claim 31, wherein said alignment device controls the speed and direction of said guide members.

33. The vertebral body preparation device of claim 31, wherein said alignment device comprises a gear.

34. The vertebral body preparation device of claim 31, wherein said alignment device comprises a connecting rod.

35. The vertebral body preparation device of claim 31, wherein said alignment device comprises a band connected between a pair of pulleys, wherein each pulley is connected to a respective guide member.
